(19) United States
(12) Patent Application Publication
(10) Pub. No.: US 2024/0325764 A1
(43) Pub. Date: Oct. 3, 2024

(54) IMPLANTABLE MEDICAL DEVICE WITH FLEX CIRCUIT

(71) Applicant: Cardiac Pacemakers, Inc., St. Paul, MN (US)

(72) Inventor: James Michael English, Cahir, Co.Tipperary (IE)

(73) Assignee: Cardiac Pacemakers, Inc., St. Paul, MN (US)

(21) Appl. No.: 18/615,244

(22) Filed: Mar. 25, 2024

Related U.S. Application Data
(60) Provisional application No. 63/454,840, filed on Mar. 27, 2023.

(51) Int. Cl. A61N 1/375

(52) U.S. Cl. CPC A61N 1/3758; A61N 1/3754

(57) ABSTRACT

Methods of manufacturing an implantable medical device, and devices resulting from such methods. A printed circuit board assembly is made, and a first portion of encapsulant is applied to at least a portion of the printed circuit board assembly. A flex circuit is attached, such as by soldering, to the printed circuit board assembly. The flex circuit is then secured to the first portion of encapsulant, prior to applying a second portion of encapsulant.

### CROSS-REFERENCE TO RELATED APPLICATIONS

This application claims the benefit of U.S. Provisional Patent Application Ser. No. 63/454,840, filed Mar. 27, 2023, which is incorporated herein by reference.

### BACKGROUND

Implantable medical devices serve a variety of therapeutic and diagnostic purposes. Many such devices include electronic circuits, power supplies and other components. Devices are often provided within an electrically conductive housing. Some such devices, such as implantable defibrillators, are configured to hold high voltages on relatively large capacitors, for example, up to 100 microfarads, for delivery of high energy outputs in the range of up to 40 Joules, for a transvenous system, or up to 80 Joules for a subcutaneous defibrillator. Shielding is often provided to prevent shorting between the electrically conductive housing and internal componentry during high energy shocks and/or during preparation for such shocks. One solution is a shield provided as a flexible circuit, such as shown in US PG Pat. Pub. No. 20103005654, titled ELECTROMAGNETIC INTERFERENCE SHIELDING IN AN IMPLANTABLE MEDICAL DEVICE.

Implantable defibrillators need also to be able to dissipate energy relatively quickly, without creating excessively localized heat. One solution is to provide a dump resistor on a flexible circuit shield, as shown in US PG Pat. Pub. No. 20160287865, titled IMPLANTABLE MEDICAL DEVICES HAVING FLEXIBLE ELECTROMAGNETIC INTERFERENCE AND DUMP RESISTOR SHIELDS. It is desired to have new and/or alternative methods for including such shields or dump resistors in implantable devices.

### Overview

The present inventors have recognized, among other things, that a problem to be solved is the need for new and/or alternative assembly methods to provide a flexible shield inside of a medical device. In particular, prior assembly methods such as in US PG Pat. Pub. Nos. 20100305654 and 20160287865 typically use the outer canister halves of the device to hold down the flexible shields or dump resistors. A frame may also be used. During final assembly, the flex circuit can simply be pushed into position prior to welding the implantable device housing shut.

However, newer methods of constructing an implantable medical device include the use of an encapsulant layer to provide mechanical (vibration) protection to components in the device as well as offering thermal and electrical isolation. See, for example, US PG Pub. No. 2022047876, titled IMPLANTABLE MEDICAL DEVICE WITH RELATIVE MOTION CONTROL, the disclosure of which is incorporated herein by reference. With this approach, selective deposits of an encapsulant are applied to portions of the operational circuitry of the implantable medical device. The flexible dump resistor or shield needs to be controlled during processing in order to ensure appropriate positioning of the encapsulant as well as the flexible dump resistor.

A first illustrative and non-limiting example takes the form of a method of assembling an implantable medical device having a flexible circuit and a plurality of additional internal components, the method comprising: assembling at least two of the additional internal components together in an assembly; depositing a first layer of encapsulant material over at least part of at least one of the additional internal components of the assembly; securing the flexible circuit to the assembly at a flexible circuit tab; attaching the flexible circuit to the first layer of encapsulant material; and applying a second layer of encapsulant material over one or more of the assembly or the flexible circuit.

Additionally or alternatively, attaching the flexible circuit to the first layer of encapsulant material comprises reflowing at least a portion of the first layer of encapsulant material. Additionally or alternatively, reflowing is achieved by applying energy from a laser welder. Additionally or alternatively, reflowing is achieved by applying heat by contact. Additionally or alternatively, the flexible circuit has an outer edge, and the step of reflowing at least a portion of the first layer of encapsulant material is performed at the outer edge of the flexible circuit to cause a portion of the first layer of encapsulant material to flow over the outer edge of the flexible circuit.

Additionally or alternatively, the flexible circuit has an outer edge, and the step of securing the flexible circuit to the first layer of encapsulant material is performed at the outer edge of the flexible circuit.

Additionally or alternatively, the assembly includes a printed circuit board and a plurality of electronic components.

Additionally or alternatively, the flexible circuit comprises a dump resistor for dumping excess energy of the implantable medical device.

Additionally or alternatively, the flexible circuit comprises an electromagnetic interference shield.

Additionally or alternatively, the flexible circuit comprises both an electromagnetic interference shield and a dump resistor.

Another illustrative and non-limiting example takes the form of an implantable medical device comprising: a flexible circuit having a flexible circuit tab; a plurality of additional internal electrical components, wherein at least two of the additional internal components are mechanically and electrically coupled together in an assembly; a first layer of encapsulant material deposited over at least part of at least one of the additional internal components of the assembly; wherein the flexible circuit is coupled to the assembly using the flexible circuit tab and is attached to the first layer of encapsulant material; and a second layer of encapsulant material applied over one or more of the assembly or the flexible circuit.

Additionally or alternatively, the flexible circuit is attached to the first layer of encapsulant material by reflowing at least a portion of the first layer of encapsulant material. Additionally or alternatively, the portion of the first layer of encapsulant material is reflowed by applying energy from a laser welder. Additionally or alternatively, the portion of the first layer of encapsulant material is reflowed by applying heat by contact. Additionally or alternatively, the flexible circuit has an outer edge, and the reflowed portion of the first layer of encapsulant material covers the outer edge of the flexible circuit.

Additionally or alternatively, the flexible circuit has an outer edge, and the first layer of encapsulant material covers at least a part of the outer edge of the flexible circuit.

Additionally or alternatively, the assembly includes a printed circuit board and a plurality of electronic components.

Additionally or alternatively, the flexible circuit comprises a dump resistor for dumping excess energy of the implantable medical device.

Additionally or alternatively, the flexible circuit comprises an electromagnetic interference shield.

Additionally or alternatively, the flexible circuit comprises both an electromagnetic interference shield and a dump resistor.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an implantable medical device without a header;

FIGS. 2-3 show partial assemblies of an implantable medical device;

FIG. 4 shows an illustrative flex circuit for an implantable medical device;

FIGS. 5-6 illustrate assembly of an implantable medical device; and

FIG. 7 is a block diagram for a process flow.

### DETAILED DESCRIPTION

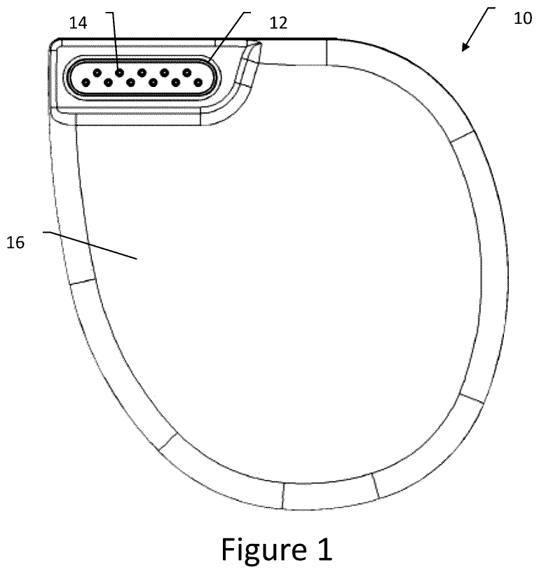

FIG. 1 shows an implantable medical device (IMD) without a header. The IMD 10 may be, for example and without limitation, an implantable defibrillator. The present invention may be used for any IMD. IMDs may be, for example and without limitation, pacemakers, defibrillators, cardiac monitors, cardiac resynchronization therapy devices, cardiac assist devices, neurostimulators, neuromodulators, spinal cord stimulators, sacral, occipital, and/or Vagus nerve stimulators, deep brain stimulation systems, or other systems. Some devices may store a therapeutic substance, such as a drug, if the IMD is a drug pump, or insulin, if the IMD is an insulin source. A reservoir for a therapeutic substance may be refillable, if desired. Devices may have a rechargeable battery and associated battery charging circuitry, or may use a non-rechargeable battery.

The IMD 10 includes a header region 12, having feedthrough pins 14 for electrical coupling via a header (not shown, but well known in the art) to a lead, for example. An electrically conductive housing 16 provides an hermetically sealed enclosure of the IMD 10. Rather than a header region with feedthrough pints 14, other outputs may be provided including a cannula for a drug pump, for example and without limitation to a specific device.

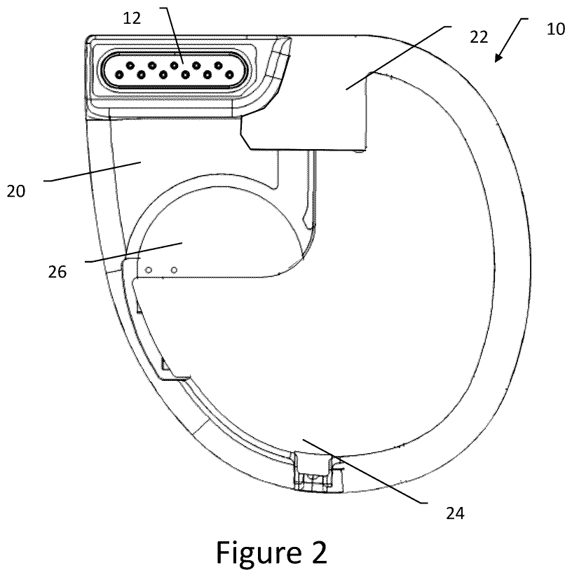

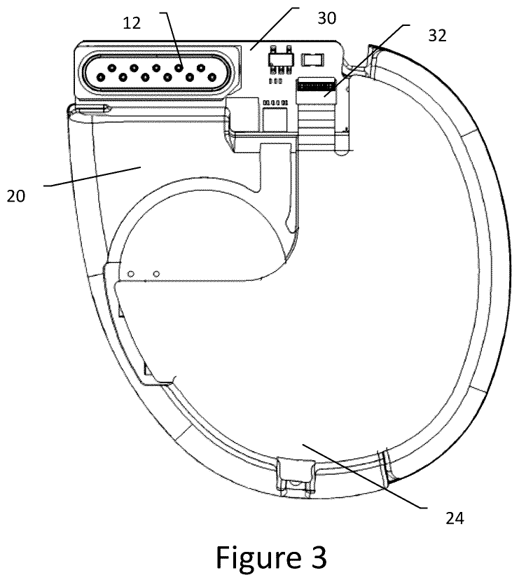

FIGS. 2-3 show partial assemblies of an implantable medical device. Here, a portion of the electrically conducive housing 16 (FIG. 1) has been removed so that inner contents can be observed. The header region 12 remains visible. An encapsulant has been used in this assembly, with a first encapsulant portion 20, and a second encapsulant portion 22. Illustratively, the first encapsulant portion 20 may be applied first, to cover and/or hold in place certain components of the operational circuitry of the IMD. As used herein, “operational circuitry” may refer to any of the electrical components used within an IMD, such as batteries, capacitors, resistors, discrete components (operational amplifiers for example), memories, application specific integrated circuits (ASIC), crystals, oscillators, inductors, microprocessors, microcontrollers, transducers, accelerometers, pumps, transformers, etc. that may be found in an IMD such as those listed above. Later in the assembly of the IMD 10, the second encapsulant portion 22 may be applied to additionally hold or secure componentry.

In this example, a flex circuit is shown at 24, and may take the form of a resistor and/or shield, such as disclosed in US PG Pat. Pub. Nos. 20100305654 and 20160287865, for example and without limitation. A piezo-transducer is shown as well at 26, such as may be used for a speaker/annunciator to issue patient alerts in an IMD 10. In an illustrative example, the partial assembly shown in FIG. 2 is secured in the position shown by the second encapsulant portion 22, which abuts and/or overlaps an exterior edge of the flex circuit 24 once deposited.

FIG. 3 illustrates the subassembly of FIG. 2, but without the second encapsulant portion present. As can be seen, a tab 32 of the flex circuit 24 is attached to a printed circuit board assembly (PCBA) 30, which carries additional electronic components, such as one or more ASICs, memory, capacitors, resistors, discrete components (operational amplifiers and the like, for example), and a microcontroller. The PCBA 30 may couple to the feedthrough 12 using, for example, a ferrule as described in US PG Pub. No. 20220288401, titled IMPLANTABLE MEDICAL DEVICE WITH FEEDTHROUGH ANTENNA GROUND STRUCTURE, if desired. Without the second encapsulant portion present in FIG. 3, the flex circuit 24 is not secured down to the rest of the IMD contents, except at the tab 32.

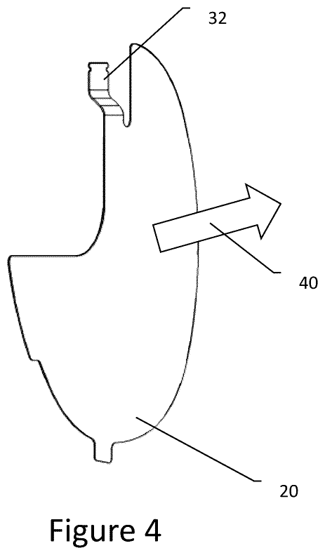

FIG. 4 shows an illustrative flex circuit for an implantable medical device. The tab is shown at 32, while the main portion of the flex circuit is shown at 20. In an example, the flex circuit may carry a plurality of traces that form a large area resistor, which can be used to dissipate high voltages stored on, for example, the high voltage capacitors of an implantable defibrillator in the event that internal discharge of the capacitors is required. Internal discharge may occur, for example, due to periodic capacitor reformation (which prevents electrical degradation in the capacitors), or if the capacitors are charged for a therapy output that is never delivered (which may happen due to spontaneous conversion of arrhythmia, or if malsensing causes inappropriate charging and is resolved before shock). The flex circuit may be an electromagnetic interference (EMI) shield as disclosed in US PG Pat. Pub. No. 20100305654, for example, or may combine both EMI shielding and a dump resistor as in US PG Pat. Pub. No. 20160287865, for example.

When the tab 32 of flex circuit 20 is secured to a PCBA as shown in FIG. 3, without other portions of the flex circuit 20 secured to something, the tendency will be to flex in direction 40, out and away from the rest of the IMD contents. This may create problems during subsequent processing and manufacturing steps; even if a fixture is used to hold the flex circuit 20 down, the presence of a fixture adds expense and other challenges to manufacturing processes.

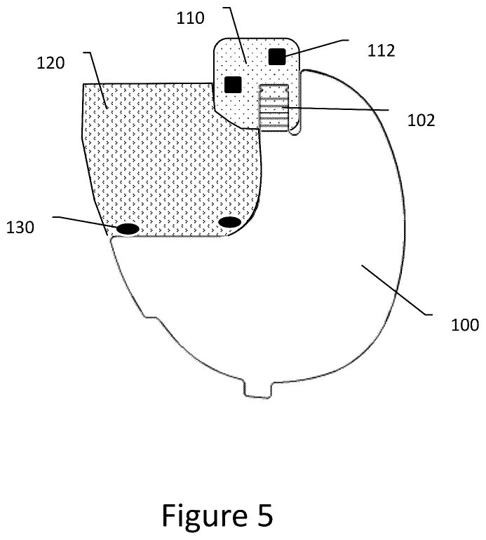

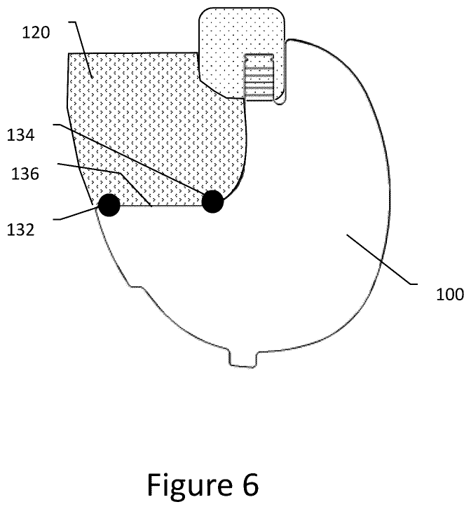

FIGS. 5-6 illustrate assembly of an implantable medical device. FIG. 5 illustrates how a flex circuit 100 can be secured via tab 102 to a PCBA 110 carrying a plurality of components 112. A first encapsulant portion 120 can be applied after the tab 102 is secured. Alternatively, the first encapsulant portion 120 may already be present when the tab 102 is secured. The first encapsulant portion 120 has, in this example, one or more bumps or ridges as illustrated at 130 of excess encapsulant.

As shown in FIG. 6, the bumps 130 are then used to secure the flex circuit 100 down at select locations, such as 132 and 134 as shown. In an example, heat is used, such as by the application energy by, for example, a laser welder, a contact welder, or the heated tip of a soldering iron, to cause reflow of the first encapsulant portion 120. Pressing the reflow of the first encapsulant portion over the outer edge 136 of the flex circuit can be used to extend the first encapsulant over the outer edge 136 of the flex circuit.

In another illustrative example, rather than reflow, additional material may be placed at locations 132 and/or 134, such as by dispensing a desired material, which may be the same as the encapsulant (illustrative materials are identified, for example, in US PG Pub. No. 2022047876, titled IMPLANTABLE MEDICAL DEVICE WITH RELATIVE MOTION CONTROL, the disclosure of which is incorporated herein by reference). Other materials, such as an adhesive, may be applied instead at desired locations.

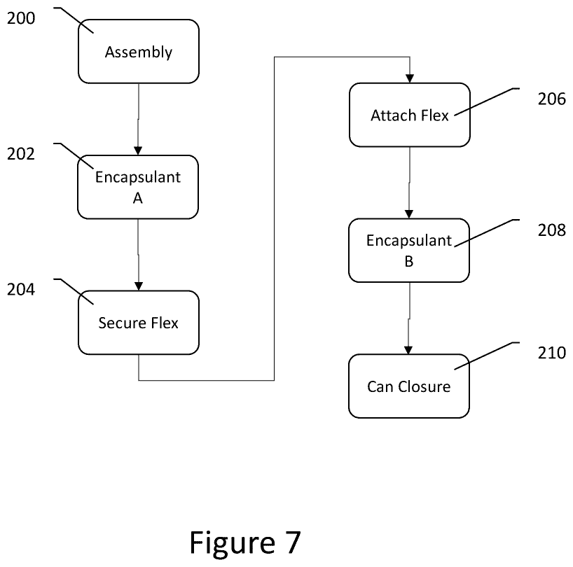

FIG. 7 is a block diagram for a process flow. An IMD may be manufactured in this example by assembly 200 of a plurality of components, such as the PCBA having thereon any of the electronic componentry identified above. For example, at least first and second (that is, at least two) electrical components may be mechanically and/or electrically coupled together such as by soldering, welding, and/or adhesive into an assembly. A first encapsulant portion is then applied, as indicated at 202 and designated Encapsulant A. A flex circuit, which may be a dump resistor, EMI shield, or combination thereof, is then secured to the assembly, as indicated at 204. Step 204 may include electrical coupling the flex circuit to the circuitry assembled in block 200, such as by electrical and mechanical attachment (often by soldering) to the PCBA, as an example. The flex circuit is then attached at 206 to the Encapsulant A. A second encapsulant portion is then applied, as indicated at 208 and designated as Encapsulant B. Finally, the IMD canister is closed, as indicated at 210.

Additional steps may occur throughout the process flow. For example, other circuitry (batteries, capacitors, feedthrough, header, antenna, etc.) may be attached to or otherwise added to the assembly. Various steps of baking, annealing, strain relief, sterilization, cleaning, etc. may occur as well. Electrical and/or functional testing steps may be added wherever appropriate to the particular build. The illustration of FIG. 7 may include any of these further procedures, as needed.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of assembling an implantable medical device having a flexible circuit and a plurality of additional internal components, the method comprising:
assembling at least two of the additional internal components together in an assembly;
depositing a first layer of encapsulant material over at least part of at least one of the additional internal components of the assembly;
securing the flexible circuit to the assembly at a flexible circuit tab;
attaching the flexible circuit to the first layer of encapsulant material; and
applying a second layer of encapsulant material over one or more of the assembly or the flexible circuit.

2. The method of claim 1 wherein attaching the flexible circuit to the first layer of encapsulant material comprises reflowing at least a portion of the first layer of encapsulant material.

3. The method of claim 2 wherein reflowing is achieved by applying energy from a laser welder.

4. The method of claim 2 wherein reflowing is achieved by applying heat by contact.

5. The method of claim 2, wherein the flexible circuit has an outer edge, and the step of reflowing at least a portion of the first layer of encapsulant material is performed at the outer edge of the flexible circuit to cause a portion of the first layer of encapsulant material to flow over the outer edge of the flexible circuit.

6. The method of claim 1, wherein the flexible circuit has an outer edge, and the step of securing the flexible circuit to the first layer of encapsulant material is performed at the outer edge of the flexible circuit.

7. The method of claim 1 wherein the assembly includes a printed circuit board and a plurality of electronic components.

8. The method of claim 1 wherein the flexible circuit comprises a dump resistor for dumping excess energy of the implantable medical device.

9. The method of claim 1 wherein the flexible circuit comprises an electromagnetic interference shield.

10. The method of claim 1 wherein the flexible circuit comprises both an electromagnetic interference shield and a dump resistor.

11. An implantable medical device comprising:
a flexible circuit having a flexible circuit tab;
a plurality of additional internal electrical components, wherein at least two of the additional internal components are mechanically and electrically coupled together in an assembly;
a first layer of encapsulant material deposited over at least part of at least one of the additional internal components of the assembly;
wherein the flexible circuit is coupled to the assembly using the flexible circuit tab and is attached to the first layer of encapsulant material; and
a second layer of encapsulant material applied over one or more of the assembly or the flexible circuit.

12. The implantable medical device of claim 11, wherein the flexible circuit is attached to the first layer of encapsulant material by reflowing at least a portion of the first layer of encapsulant material.

13. The implantable medical device of claim 12, wherein the portion of the first layer of encapsulant material is reflowed by applying energy from a laser welder.

14. The implantable medical device of claim 12, wherein the portion of the first layer of encapsulant material is reflowed by applying heat by contact.

15. The implantable medical device of claim 12, wherein the flexible circuit has an outer edge, and the reflowed portion of the first layer of encapsulant material covers the outer edge of the flexible circuit.

16. The implantable medical device of claim 11, wherein the flexible circuit has an outer edge, and the first layer of encapsulant material covers at least a part of the outer edge of the flexible circuit.

17. The implantable medical device of claim 11, wherein the assembly includes a printed circuit board and a plurality of electronic components.

18. The implantable medical device of claim 11, wherein the flex circuit comprises a dump resistor for dumping excess energy of the implantable medical device.

19. The implantable medical device of claim 11, wherein the flex circuit comprises an electromagnetic interference shield.

20. The implantable medical device of claim 11, wherein the flex circuit comprises both an electromagnetic interference shield and a dump resistor.
